(19) United States
(12) Patent Application Publication
(10) Pub. No.: US 2024/0350222 A1
(43) Pub. Date: Oct. 24, 2024

(54) NON-ADHESIVE MEDICAL ATTACHMENT ARTICLES USING MICRONEEDLES

(71) Applicant: SOLVENTUM INTELLECTUAL PROPERTIES COMPANY, Maplewood, MN (US)

(72) Inventors: Del R. Lawson, Cottage Grove, MN (US); Audrey A. Sherman, Woodbury, MN (US); Matthew M. Cooper, Lake Elmo, MN (US); Jeremy K. Larsen, Farmington, MN (US); Bret W. Ludwig, Oakdale, MN (US); Brian J. Gates, Osceola, WI (US); Alexander J. Huffman, St. Paul, MN (US); Kyle C. Picha, Maplewood, MN (US)

(21) Appl. No.: 18/683,021

(22) PCT Filed: Sep. 9, 2022
(86) PCT No.: PCT/IB2022/058519

§ 371 Date: Feb. 12, 2024

Related U.S. Application Data
(60) Provisional application No. 63/246,159, filed on Sep. 20, 2021.

(51) Int. Cl. A61B 50/20; A61B 90/00

(52) U.S. Cl. CPC A61B 50/20; A61B 90/08

(57) ABSTRACT

Non-adhesive medical attachment articles include a first rotationally distinct segment (110), where the first rotational segment has a circular or semi-circular array of angled microneedles (111), and a second rotationally distinct segment, (120) with a second sequential circular or semi-circular array of angled microneedles (121). A communication member (130) connects the two rotationally distinct segments. The arrays of microneedles are angled in opposite directions, and the segments rotate in opposite directions.

### SUMMARY

Disclosed herein are non-adhesive medical attachment articles, medical constructions that include these non-adhesive medical attachment articles, and methods of using these non-adhesive medical attachment articles in medical constructions.

Disclosed herein are composite medical articles that comprise an attachment article and a medical device attached to at least a portion of the attachment article. In some embodiments, the attachment article comprises a discontinuous base layer comprising distinct segments with a first major surface and a second major surface. The article further comprises a first rotationally distinct segment, where the first rotational segment is circular or semicircular, has a first major surface and a second major surface, and the second major surface is attached to a portion of a segment of the discontinuous base layer. The first major surface comprises a first sequential circular or semi-circular array of angled microneedles. The article also comprises a second rotationally distinct segment, where the second rotational segment is circular or semicircular, has a first major surface and a second major surface, and the second major surface is attached to a portion of a segment of the discontinuous base layer. The first major surface comprises a second sequential circular or semi-circular array of angled microneedles. A communication member is disposed between and at least in contact with the first rotationally distinct segment and the second rotationally distinct segment. The angle of a microneedle of the first array of microneedles, relative to a point on the first major surface of the first rotational segment is such that the point, the point of attachment of the microneedle to the first major surface of the first rotational segment, and the tip of the microneedle forms an angle of 40-80°, and the angle of a microneedle of the second array of microneedles, relative to the same point on the first major surface of the first rotational segment is such that the point, the point of attachment of the microneedle to the first major surface of the second rotational segment, and the tip of the microneedle forms an angle of 140-100°.

Also disclosed are medical constructions. In some embodiments, the medical construction comprises a substrate comprising mammalian skin, an attachment article as described above, where only the microneedles of the attachment article are in contact with the substrate surface, and a device attached to at least a portion of the attachment article. The medical construction has a void space between the substrate surface and the first major surfaces of the first and second rotational segments of the attachment article.

Also disclosed are methods of attaching a medical device to mammalian skin comprising providing a substrate comprising mammalian skin, providing a composite medical article, as described above, contacting at least some of the angled microneedles of the attachment article to the surface of the substrate, and rotating the first rotationally distinct segment of the attachment device in a direction that causes at least some of the angled microneedles of the first sequential circular or semi-circular array to penetrate the surface of the substrate.

### BRIEF DESCRIPTION OF THE DRAWINGS

The present application may be more completely understood in consideration of the following detailed description of various embodiments of the disclosure in connection with the accompanying drawings.

FIG. 1A is a bottom view of the rotational segments of an article of this disclosure.

FIG. 1B is a top view of the rotational segments of the article of FIG. 1A.

FIG. 2A is a side view of the rotational segments of another article of this disclosure.

FIG. 2B is a top view of the rotational segments of the article of FIG. 2A.

FIG. 3 shows a top view of an article of this disclosure.

FIG. 4 shows a top view of another article of this disclosure.

FIG. 5 shows a side view of an angled microneedle of the current disclosure.

FIG. 6 shows a side view of another angled microneedle of the current disclosure.

In the following description of the illustrated embodiments, reference is made to the accompanying drawings, in which is shown by way of illustration, various embodiments in which the disclosure may be practiced. It is to be understood that the embodiments may be utilized and structural changes may be made without departing from the scope of the present disclosure. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

The attachment of medical articles and devices to skin surfaces is growing in importance. In some cases, the attachment is for a short duration such as hours, and in other cases the attachment is for a long duration such as weeks or even months. The attachment method has a wide range of requirements, not only to hold the article or device in place, but also to be removable when desired, to not damage the skin, and to not cause skin irritation or damage when worn. Many of the solutions developed involve the use of adhesives for skin attachment. In some instances, the adhesive is a pressure sensitive adhesive, in other instances the adhesive is a gel. The choice of adhesive can depend upon the desired use. These adhesives can be gels such as those that hold sensors to the skin or tapes which are layers of pressure sensitive adhesive on a backing. The use of adhesive products in the medical industry has long been prevalent and is increasing. However, while adhesives and adhesive articles have shown themselves to be very useful for medical applications, there are also issues in the use of adhesives and adhesive articles. Therefore, there remains a need for methods of attachment of medical articles and devices to skin surfaces that do not use or fully rely upon adhesive layers for attachment.

As mentioned above, while adhesives and adhesive articles have shown themselves to be very useful for medical applications, there are also issues in the use of adhesives and adhesive articles. Medical adhesive-related skin injury (MARSI) has a significant negative impact on patient safety. Skin injury related to medical adhesive usage is a prevalent but under-recognized complication that occurs across all care settings and among all age groups. In addition, treating skin damage is costly in terms of service provision, time, and additional treatments and supplies.

Skin Injury occurs when the superficial layers of the skin are removed along with the medical adhesive product, which not only affects skin integrity but can cause pain and the risk of infection, increase wound size, and delay healing, all of which reduce patients' quality of life.

In this disclosure, attachment articles are described that use arrays of microneedles to attach medical articles and devices to substrate surfaces, especially mammalian skin. The angled microneedles are arranged in a circular or semi-circular array such that the microneedles can be rotationally attached to the surface. These attachment articles not only firmly adhere to the surface, but also form a gap between the attachment article and the surface. This prevents the build up of moisture under the attachment article and also permits cleaning of the surface under the attachment article while the attachment article remains adhered. This is different from adhesive attachment where the adhesive is in direct contact with the skin surface.

Also disclosed are medical constructions that include a substrate surface such as mammalian skin and an attachment article adhered to the substrate surface. The attachment article can be used to attach a wide range of medical articles and devices to the substrate surface. Methods for attaching articles to substrate surfaces are also disclosed.

The term “adhesive” as used herein refers to polymeric compositions useful to adhere together two adherends. Examples of adhesives are pressure sensitive adhesives and gel adhesives.

Pressure sensitive adhesive compositions are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as pressure sensitive adhesives are polymers designed and formulated to exhibit the requisite viscoelastic properties resulting in a desired balance of tack, peel adhesion, and shear holding power. Obtaining the proper balance of properties is not a simple process.

As used herein, the term “gel adhesive” refers to a tacky semi-solid crosslinked matrix containing a liquid or a fluid that is capable of adhering to one or more substrates. The gel adhesives may have some properties in common with pressure sensitive adhesives, but they are not pressure sensitive adhesives. “Hydrogel adhesives” are gel adhesives that have water as the fluid contained within the crosslinked matrix.

The terms “polymer” and “macromolecule” are used herein consistent with their common usage in chemistry. Polymers and macromolecules are composed of many repeated subunits. As used herein, the term “macromolecule” is used to describe a group attached to a monomer that has multiple repeating units. The term “polymer” is used to describe the resultant material formed from a polymerization reaction.

The term “microneedles” as used herein refers to skin anchoring microstructural protrusions with points. The microneedles may be solid or they may be hollow to permit the transmission of fluids.

Disclosed herein are composite medical articles. The composite medical articles comprise an attachment article and a device attached to the attachment article. A wide range 10 of composite medical articles are disclosed.

In some embodiments, the attachment article comprises a discontinuous base layer comprising distinct segments with a first major surface and a second major surface. The attachment article further comprises a first rotationally distinct segment and second rotationally distinct segment. The first rotational segment is circular or semicircular and has a first major surface and a second major surface, where the second major surface is attached to a portion of a segment of the discontinuous base layer, and the first major surface comprises a first sequential circular or semi-circular array of angled microneedles. The second rotationally distinct segment is circular or semicircular, has a first major surface and a second major surface, where the second major surface is attached to a portion of a segment of the discontinuous base layer, and the first major surface comprises a second sequential circular or semi-circular array of angled microneedles. A communication member is disposed between and at least in contact with the first rotationally distinct segment and the second rotationally distinct segment. The angled microneedles of the first array of microneedles are angled in a direction opposite to the direction of the angled microneedles of the second array of microneedles. Stated another way, the angle of a microneedle of the first array of microneedles, relative to a point on the first major surface of the first rotational segment is such that the point, the point of attachment of the microneedle to the first major surface of the first rotational segment, and the tip of the microneedle forms an angle of 40-80°, and the angle of a microneedle of the second array of microneedles, relative to the same point on the first major surface of the first rotational segment is such that the point, the point of attachment of the microneedle to the first major surface of the second rotational segment, and the tip of the microneedle forms an angle of 140-100°.

The rotational aspect of the first sequential circular or semi-circular array of microneedles permits twist on, twist off attachment. By this it is meant that because of the circular or semi-circular array of microneedles, by twisting the array the microneedles penetrate substrates like mammalian skin in a controlled way. The twisting permits the microneedles to penetrate the substrate surface, but not so deep as to cause pain. Because the microneedles are forced into the substrate surface by twisting, the depth of penetration of the microneedles into the substrate surface is carefully controlled.

A wide range of microneedles are suitable use in the first sequential circular or semi-circular array of current articles. A wide variety of materials are suitable for use in the microneedles. In some embodiments, the microneedles comprise plastics, metals, absorbable materials, or a combination thereof. Suitable plastics include polyolefinic materials, polyesters, polyurethanes, and the like. Suitable metals include stainless steel, titanium, and Nitinol (a nickel/titanium alloy). Absorbable materials include materials used to form absorbable sutures, such as polyglycolide (such as the commercial materials DEXON, DEXON II, DEXON S), poly(glycolide/lactide) random copolymer (such as the commercial materials VICRYL and VICRYL PLUS), poly-p-dioxanone (such as the commercial materials PDS, PDSII, and PDS PLUS), poly(glycolide/trimethylene carbonate) block copolymer (such as the commercial material MAXON), poly(glycolide/s-caprolactone) (such as the commercial materials MONOCRYL, and MONOCRYL PLUS), poly(gycolide/p-dioxanone/trimethylene carbonate)triblock copolymer (such as the commercial material BIOSYN), poly(glycolide/ε-caprolactone/trimethylene carbonate) triblock copolymer (such as the commercial material MONOSYN), poly(glycolide/L-lactide/ε-caprolactone/trimethylene carbonate) teriblock copolymer (such as the commercial material CAPROSYN), and 100% poly-L-lactide (such as the commercial material ORTHODEK).

A wide variety of shapes are suitable for the microneedles. In some embodiments, the microneedles comprise tapered cones comprising a point. In some embodiments, the microneedles further comprise at least one barb. The barbs can assist in securing the microneedles after the microneedles penetrate the substrate surface. Additionally, in some embodiments, the microneedles comprise a stopping portion to limit the depth that the microneedle can penetrate the substrate surface. The stopping portion may be a region of greater thickness, or a protrusion extending from the surface of the microneedle.

In some embodiments, all of the microneedles in the first array of angled microneedles are of essentially the same shape and are of the same composition. In other embodiments, some of the microneedles in the first array of angled microneedles are different in shape, composition, configuration, or a combination thereof from the majority of the microneedles in the first array of microneedles. In some embodiments, the different microneedles are hollow permitting the flow of fluids through the microneedles, are conductive permitting electric monitoring through the microneedle, are bioabsorbable, or a combination thereof.

The second sequential array of angled microneedles comprises a second circular or semi-circular array. The second sequential array of angled microneedles comprise the materials and shapes described above. The second sequential array of angled microneedles, like the first array of angled microneedles, may all be the same, or some of the microneedles may be different in shape, composition, configuration, or a combination thereof from the majority of the microneedles in the second array of microneedles. Typically, the angled microneedles are angled in a direction that is opposite to the angle direction of the first sequential array of angled microneedles. When the first rotationally distinct segment is rotated so that the first sequential array of angled microneedles into the substrate surface, the second array of angled microneedles also penetrate the substrate surface to aid in the securement of the article.

In some embodiments, all of the first sequential array of angled microneedles, the second array of angled microneedles, or both are all bioabsorbable materials. In other embodiments, some of the first sequential array of angled microneedles, the second array of angled microneedles, or both are all bioabsorbable materials.

The second rotationally distinct segment rotates in a direction contrary to the direction of rotation of the first rotationally distinct segment. In this way, the rotation of the second rotationally distinct segment provides a second method of anchoring the article to the substrate surface. The combination of anchors angled in opposite directions provides anchoring that is resistant to applied stress in a variety of directions.

The first and second rotationally distinct segments are linked to each other by communication members. These communication members maintain the relative positions of the first and second rotationally distinct segments while permitting the segments to rotate relative to each other. In some embodiments, the communication member comprises a compliant member or a rolling member. Compliant members are flexible and semi-rigid members that are attached to one of the rotationally distinct segments and are held in tension against the other rotationally distinct segment. The compliant member may be, for example, a spring or a flexible bar. In these embodiments, the compliant member is attached to rotationally distinct segment A and is held in tension against rotationally distinct segment B. In these embodiments, the edge of rotationally distinct segment B may contain a groove to help hold the compliant member in tension, or it may contain features such as protrusions or ditches to assist the compliant member in tension. In this way, when the first rotationally distinct segment is rotated, (the first rotationally distinct segment may be segment A or segment B) the compliant member is released from tension and when rotation of the first rotationally distinct segment is stopped tension is restored to the compliant member to hold the rotated configuration in place. When the second rotationally distinct segment is rotated, the compliant member again is released from tension to permit the rotation and when the rotation of the first rotationally distinct segment is stopped, the tension is restored to the compliant member to hold the rotated configuration in place.

In other embodiments, the communication member comprises a rolling member. The rolling member is in contact with both rotationally distinct members but is not attached to either member. In some embodiments, at least one edge, or both of the edges of the rotationally distinct segments, contain grooves or channels. These grooves or channels hold the rolling member in place and also permit the rotation of the rolling member. Examples of suitable rolling members include ball bearings and discs. As with the compliant member embodiments described above, in the rolling member embodiments, the edges of the rotationally distinct segments can contain optional protrusions or ditches to form stopping points to hinder the rotation of the rolling members. As with the compliant member embodiments described above, when the first rotationally distinct segment is rotated, the rolling member rolls in a first direction. When the second rotationally distinct segment is rotated, the rolling member rolls in a second direction.

Other communication members can be utilized as long as the communication members provide the dual functions of linking the two rotationally distinct segments together and also permitting the free rotation of the rotationally distinct segments.

The rotation of the first and second rotationally distinct segments can be carried out in a variety of ways. In some embodiments, the user of the device grasps the segments and rotates. In other embodiments, a handle can be used to carry out the rotation on one or both of the rotationally distinct segments. A handle is more easily grasped and permits more facile rotation of the rotationally distinct segments. The handle can have a wide variety of shapes and size and can be permanently attached to the rotationally distinct segment or segments or it may be attachable and removable from the rotationally distinct segment. As the rotationally distinct segments are concentric, it may be desirable to use a handle to rotate the inner rotationally distinct segment as this segment is more difficult for a user to rotate by grasping the segment. The outer rotationally distinct segment is more accessible to the user's hand and a handle may not be necessary to permit rotation. In some embodiments, a handle can be used with both rotationally distinct segments.

An additional advantage of the currently disclosed attachment articles is that the first and second arrays of microneedles penetrate the substrate surface to a limited depth, and therefore the second major surface of the attachment article does not touch the substrate surface. Therefore, a void space is present between the substrate surface and second major surface of the attachment article. This void space is particularly desirable when the substrate surface is mammalian skin, since it provides a channel under the attachment article that permits the evaporation of sweat, limits the contact to the skin and therefore limits the possibility for skin irritation, and permits cleaning of the area of skin under the attachment article.

The composite medical article further comprises a device attached to at least a portion of the first major surface of the base layer of the attachment article. A wide range of devices are suitable. Examples of a suitable device include a sensor, a monitor, tubing, a vascular access device, a drainage catheter, a drive line device, or a wearable medical device.

Also disclosed herein are medical constructions. The medical constructions comprise a substrate comprising mammalian skin and a composite medical article attached to the substrate surface. The composite medical article comprises an attachment article and a device attached to the attachment article. Suitable composite medical articles and devices have been described above.

In some embodiments, the medical construction further comprises an optional adhesive article. Examples of adhesive articles include for example adhesive tapes. The adhesive article, while not necessary to attach the medical construction to the substrate surface, can aid in the attachment of the medical construction to the substrate surface. The adhesive article can provide additional features, especially sealing. In some embodiments, adhesive articles seal the medical construction from the outside environment.

Also disclosed herein are methods of attaching medical devices to mammalian skin. In some embodiments, the method comprises providing a substrate comprising mammalian skin, providing a composite medical article, and contacting the composite medical article to the mammalian skin. The composite medical articles are described above and comprise an attachment article and a device attached to the attachment article.

In some embodiments, the attachment article comprises a discontinuous base layer comprising distinct segments with a first major surface and a second major surface. The attachment article further comprises a first rotationally distinct segment and second rotationally distinct segment. The first rotational segment is circular or semicircular and has a first major surface and a second major surface, where the second major surface is attached to a portion of a segment of the discontinuous base layer, and the first major surface comprises a first sequential circular or semi-circular array of angled microneedles. The second rotationally distinct segment is circular or semicircular, has a first major surface and a second major surface, where the second major surface is attached to a portion of a segment of the discontinuous base layer, and the first major surface comprises a second sequential circular or semi-circular array of angled microneedles. A communication member is disposed between and at least in contact with the first rotationally distinct segment and the second rotationally distinct segment. The angled microneedles of the first array of microneedles are angled in a direction opposite to the direction of the angled microneedles of the second array of microneedles. Stated another way, the angle of a microneedle of the first array of microneedles, relative to a point on the first major surface of the first rotational segment is such that the point, the point of attachment of the microneedle to the first major surface of the first rotational segment, and the tip of the microneedle forms an angle of 40-80°, and the angle of a microneedle of the second array of microneedles, relative to the same point on the first major surface of the first rotational segment is such that the point, the point of attachment of the microneedle to the first major surface of the second rotational segment, and the tip of the microneedle forms an angle of 140-100°.

Attachment of the attachment article to the substrate surface comprises contacting at least some of the angled microneedles of the attachment article to a surface of the substrate, rotating the first rotationally distinct segment of the attachment device to press at least some of the angled microneedles of the first sequential circular or semi-circular array into the surface of the substrate.

In some embodiments, where the method further comprises rotating the second rotationally distinct segment of the attachment device in the opposite direction of the first rotationally distinct segment to press at least some of the angled microneedles of the second sequential circular array into the surface of the substrate.

The method further comprises removing the attachment article. The removal of the attachment article may be to move the article to a different location, or it may be that the attachment is no longer needed to be attached to the substrate surface. Removal can be carried out by counter-rotating the rotating the second rotationally distinct segment of the attachment device in a direction that causes at least some of the angled microneedles of the second sequential circular or semi-circular array to withdraw from the surface of the substrate; and counter-rotating the first rotationally distinct segment of the attachment device in a direction that causes at least some of the angled microneedles of the first sequential circular or semi-circular array to withdraw from the surface of the substrate. As is well understood in the medical art, removal of adhesively-based attachment articles can be very painful and can cause skin damage, whereas the current attachment articles can avoid these issues.

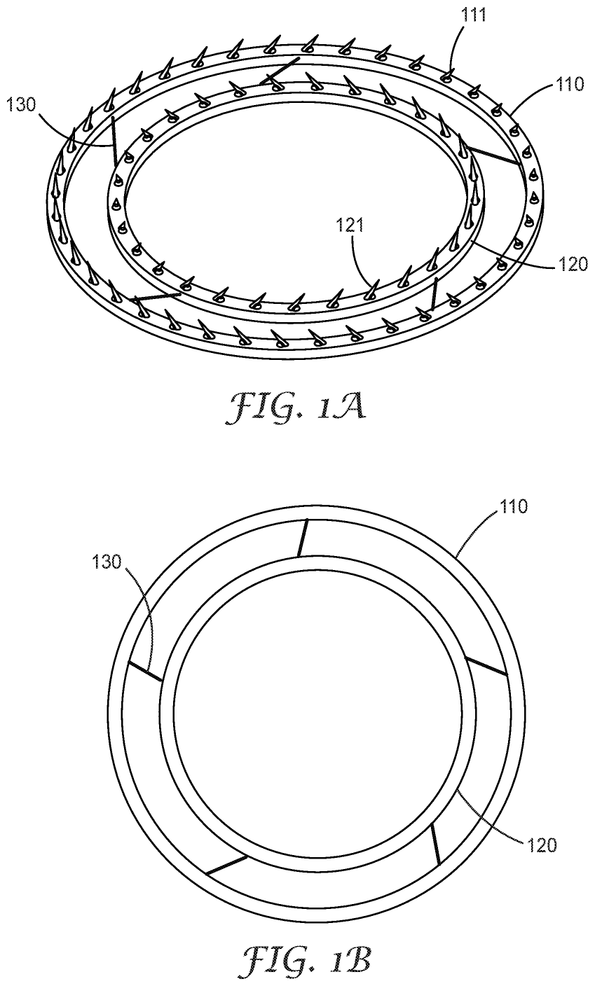

This disclosure may be further understood by reference to the figures. FIG. 1A is a bottom view of the rotational segments of an article of this disclosure. The first rotational segment 110 contains an array of angled microneedles 111, the second rotational segment 120 contains an array of angled microneedles 121. The angled microneedles 111 and 121 are angled in opposite directions. The two rotational segments are linked by compliant communication member 130. Compliant communication member 130 can be a spring or a flexible bar. The first and second rotational segments rotate in opposite directions.

FIG. 1B is a top view of the rotational segments of the article of FIG. 1A showing first rotational segment 110, second rotational segment 120, and compliant communication member 130.

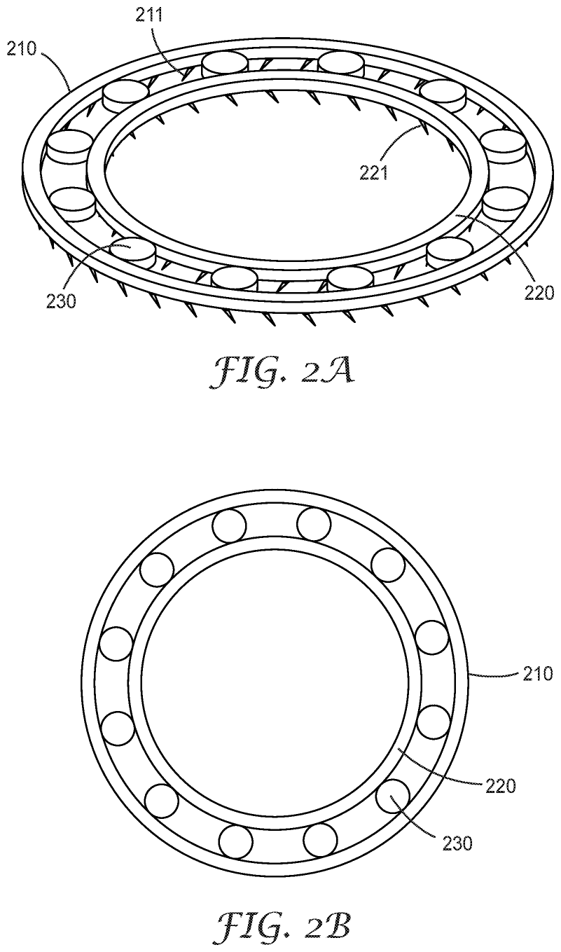

FIG. 2A is a side view of the rotational segments of an article of this disclosure. The first rotational segment 210 contains an array of angled microneedles 211, the second rotational segment 220 contains an array of angled microneedles 221. The angled microneedles 211 and 221 are angled in opposite directions. The two rotational segments are linked by rolling communication member 230. The rolling communication member can be a ball bearing or a disc. The first and second rotational segments rotate in opposite directions.

FIG. 2B is a top view of the rotational segments of the article of FIG. 2A showing first rotational segment 210, second rotational segment 220, and rolling communication member 230.

FIG. 3 shows a top view of an article of this disclosure that includes the rotational segments shown in FIGS. 1A and 1B. FIG. 3 shows first rotational segment 310, second rotational segment 320, and compliant communication member 330. Compliant communication member 330 can be a spring or a flexible bar. Discontinuous base layer 340 covers the two rotational segments. The article includes rotational handles 350 and 355 permitting a user to rotate the rotational segments. Rotational handle 350 permits rotation of the first rotational segment 310, rotational handle 355 permits rotation of the second rotational segment 320. Rotational handles 350 and 355 are detachable.

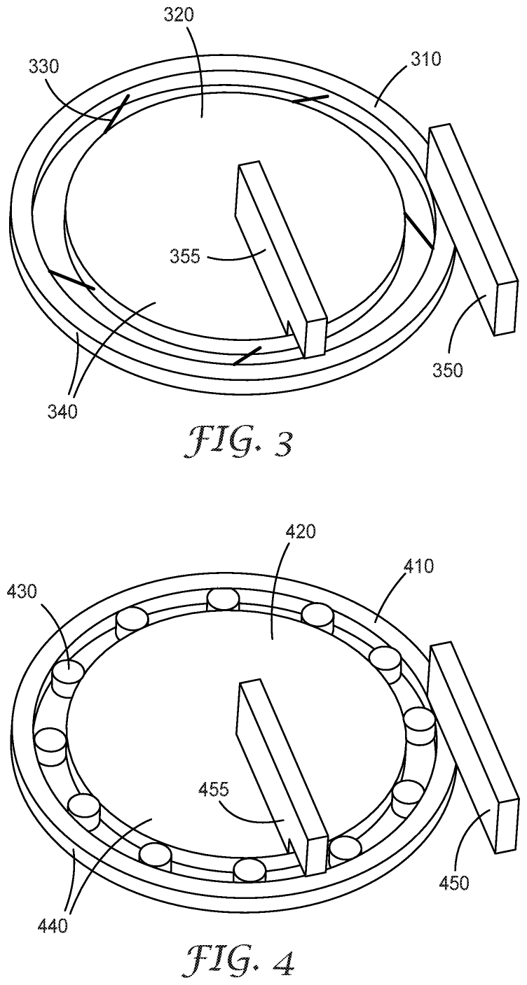

FIG. 4 shows a top view of an article of this disclosure that includes the rotational segments shown in FIGS. 2A and 2B. FIG. 4 shows first rotational segment 410, second rotational segment 420, and compliant communication member 430. Rolling communication member 430 can be a ball bearing or a disc. Discontinuous base layer 440 covers the two rotational segments. The article includes rotational handles 450 and 355 permitting a user to rotate the rotational segments. Rotational handle 450 permits rotation of the first rotational segment 410, rotational handle 455 permits rotation of the second rotational segment 420. Rotational handles 450 and 455 are detachable.

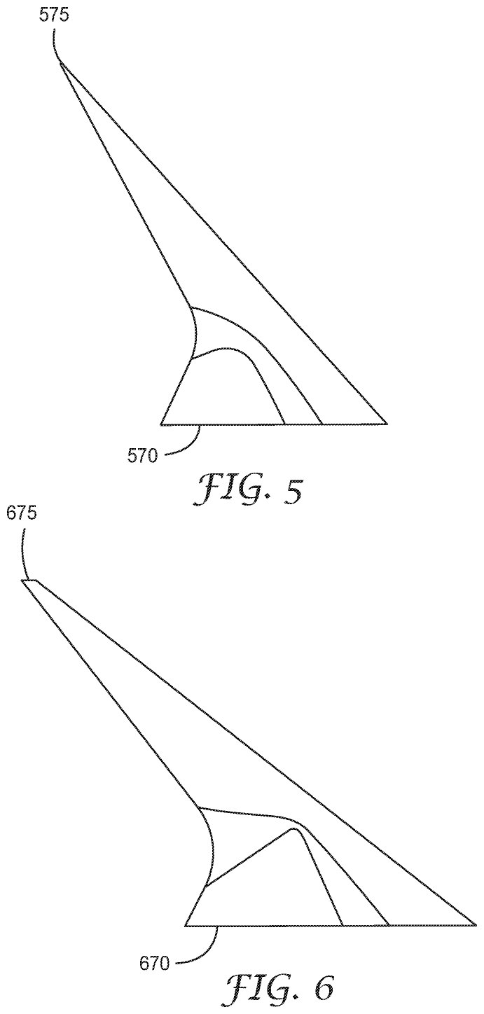

FIG. 5 shows a side view of an angled microneedle of the current disclosure. The microneedle has needle base 570 and needle tip 575.

FIG. 6 shows a side view of an angled microneedle of the current disclosure. The microneedle has needle base 670 and needle tip 675. In this microneedle, the tip 675 is an opening and the microneedle is hollow.

### Examples

[Examples section to be completed in a separate document and recombined later.]

What is claimed is:

1. A composite medical article comprising:
an attachment article comprising:
a discontinuous base layer comprising distinct segments with a first major surface and a second major surface;
a first rotationally distinct segment, where the first rotational segment is circular or semicircular, has a first major surface and a second major surface, wherein the second major surface is attached to a portion of a segment of the discontinuous base layer, and wherein the first major surface comprises a first sequential circular or semi-circular array of angled microneedles;
a second rotationally distinct segment, where the second rotational segment is circular or semicircular, has a first major surface and a second major surface, and wherein the second major surface is attached to a portion of a segment of the discontinuous base layer, and wherein the first major surface comprises a second sequential circular or semi-circular array of angled microneedles; and
a communication member disposed between and at least in contact with the first rotationally distinct segment and the second rotationally distinct segment, wherein the angle of a microneedle of the first array of microneedles, relative to a point on the first major surface of the first rotational segment is such that the point, the point of attachment of the microneedle to the first major surface of the first rotational segment, and the tip of the microneedle forms an angle of 40-80°, and the angle of a microneedle of the second array of microneedles, relative to the same point on the first major surface of the first rotational segment is such that the point, the point of attachment of the microneedle to the first major surface of the second rotational segment, and the tip of the microneedle forms an angle of 140-100°; and
a device attached to at least a portion of the first major surface of the base layer of the attachment article.

2. The composite medical article of claim 1, wherein the communication member comprises a compliant member or a rolling member.

3. The composite medical article of claim 2, wherein the communication member comprises a compliant member selected from a spring or a flexible bar, or a rolling member comprising a ball bearing or a disc.

4. The composite medical article of claim 1, wherein the device comprises a sensor, a monitor, tubing, a vascular access device, a drainage catheter, a drive line device, or a wearable medical device.

5. The composite medical article of claim 1, wherein the microneedles of the attachment article comprise tapered cones comprising a point.

6. The composite medical article of claim 1, wherein the microneedles further comprise at least one barb or a stopping portion to limit the depth that the microneedle can penetrate the substrate surface.

7. The composite medical article of claim 1, wherein some of the microneedles in the first array of angled microneedles, the second array of angled microneedles, or both are different from the majority of the angled microneedles, the difference being different in shape, configuration, composition or a combination thereof.

8. The composite medical article of claim 7, wherein the different microneedles in the microneedle array or arrays are hollow permitting the flow of fluids through the microneedles, are conductive permitting electric monitoring through the microneedle, are bioabsorbable, or a combination thereof.

9. The composite medical article of claim 1, wherein the microneedles of the first and second arrays comprise plastics, metals, absorbable materials, or a combination thereof.

10. The composite medical article of claim 1, wherein the microneedles of the first or the second arrays or both comprise absorbable materials.

11. A medical construction comprising:
a substrate comprising mammalian skin;
an attachment article attached to the substrate surface by a plurality of angled microneedles, such that only the microneedles of the attachment article are in contact with substrate surface, wherein the attachment article comprises:
a discontinuous base layer comprising distinct segments with a first major surface and a second major surface;
a first rotationally distinct segment, where the first rotational segment is circular or semicircular, has a first major surface and a second major surface, wherein the second major surface is attached to a portion of a segment of the discontinuous base layer, and wherein the first major surface comprises a first sequential circular or semi-circular array of angled microneedles;
a second rotationally distinct segment, where the second rotational segment is circular or semicircular, has a first major surface and a second major surface, and wherein the second major surface is attached to a portion of a segment of the discontinuous base layer, and wherein the first major surface comprises a second sequential circular or semi-circular array of angled microneedles; and
a communication member disposed between and at least in contact with the first rotationally distinct segment and the second rotationally distinct segment, wherein the angle of a microneedle of the first array of microneedles, relative to a point on the first major surface of the first rotational segment is such that the point, the point of attachment of the microneedle to the first major surface of the first rotational segment, and the tip of the microneedle forms an angle of 40-80°, and the angle of a microneedle of the second array of microneedles, relative to the same point on the first major surface of the first rotational segment is such that the point, the point of attachment of the microneedle to the first major surface of the second rotational segment, and the tip of the microneedle forms an angle of 140-100°; and
a device attached to at least a portion of the attachment article, wherein there is a void space between the substrate surface and the first major surfaces of the first and second rotational segments of the attachment article.

12. The medical construction of claim 11, wherein the communication member comprises a compliant member or a rolling member.

13. The medical construction of claim 12, wherein the communication member comprises a compliant member selected from a spring or a flexible bar, or a rolling member comprising a ball bearing or a disc.

14. The medical construction of claim 11, wherein the device comprises a sensor, a monitor, tubing, a vascular access device, a drainage catheter, a drive line device, or a wearable medical device.

15. The medical construction of claim 11, wherein some of the microneedles in the first array of angled microneedles, the second array of angled microneedles, or both are different from the majority of the angled microneedles, the difference being different in shape, configuration, composition or a combination thereof.

16. The medical construction of claim 15, wherein the different microneedles in the microneedle array are hollow permitting the flow of fluids through the microneedles, are conductive permitting electric monitoring through the microneedle, are bioabsorbable, or a combination thereof.

17. The medical construction of claim 11, wherein the microneedles of the first and second arrays comprise plastics, metals, absorbable materials, or a combination thereof.

18. The medical construction of claim 11, further comprising an adhesive article, wherein the adhesive article seals at least a portion of the attachment article of the medical construction from the outside environment, aids in the attachment of the medical construction to the substrate surface, or both.

19. A method of attaching a medical device to mammalian skin comprising:
providing a substrate comprising mammalian skin;
providing a composite medical article, wherein the composite medical article comprises:
an attachment article comprising:
a discontinuous base layer comprising distinct segments with a first major surface and a second major surface;
a first rotationally distinct segment, where the first rotational segment is circular or semicircular, has a first major surface and a second major surface, wherein the second major surface is attached to a portion of a segment of the discontinuous base layer, and wherein the first major surface comprises a first sequential circular or semi-circular array of angled microneedles;
a second rotationally distinct segment, where the second rotational segment is circular or semicircular, has a first major surface and a second major surface, and wherein the second major surface is attached to a portion of a segment of the discontinuous base layer, and wherein the first major surface comprises a second sequential circular or semi-circular array of angled microneedles; and
a communication member disposed between and at least in contact with the first rotationally distinct segment and the second rotationally distinct segment, wherein the angle of a microneedle of the first array of microneedles, relative to a point on the first major surface of the first rotational segment is such that the point, the point of attachment of the microneedle to the first major surface of the first rotational segment, and the tip of the microneedle forms an angle of 40-80°, and the angle of a microneedle of the second array of microneedles, relative to the same point on the first major surface of the first rotational segment is such that the point, the point of attachment of the microneedle to the first major surface of the second rotational segment, and the tip of the microneedle forms an angle of 140-100°; and
a device attached to at least a portion of the first major surface of the base layer of the attachment article;
contacting at least some of the angled microneedles of the attachment article to a surface of the substrate; and
rotating the first rotationally distinct segment of the attachment device in a direction that causes at least some of the angled microneedles of the first sequential circular or semi-circular array to penetrate the surface of the substrate.

20. The method of claim 19, further comprising rotating the second rotationally distinct segment of the attachment device in a direction that causes at least some of the angled microneedles of the second sequential circular or semi-circular array to penetrate the surface of the substrate.

21. The method of claim 20, further comprising counter-rotating the second rotationally distinct segment of the attachment device in a direction that causes at least some of the angled microneedles of the second sequential circular or semi-circular array to withdraw from the surface of the substrate; and counter-rotating the first rotationally distinct segment of the attachment device in a direction that causes at least some of the angled microneedles of the first sequential circular or semi-circular array to withdraw from the surface of the substrate.
